(19) United States
(12) Patent Application Publication
(10) Pub. No.: US 2017/0275569 A1
(43) Pub. Date: Sep. 28, 2017

(54) HAND DISHWASHING DETERGENT HAVING AN IMPROVED EFFECT AGAINST STARCH

(71) Applicant: Henkel AG & Co. KGaA, Duesseldorf (DE)

(72) Inventors: Ulrich Pegelow, Duesseldorf (DE); Detlef Buisker, Essen (DE); Timothy O'Connell, Landsberg am Lech (DE); Ines Raschke, Langenfeld (DE)

(73) Assignee: Henkel AG & Co. KGaA, Duesseldorf (DE)

(21) Appl. No.: 15/614,319

(22) Filed: Jun. 5, 2017

Related U.S. Application Data
(63) Continuation of application No. PCT/EP2015/078400, filed on Dec. 2, 2015.

(30) Foreign Application Priority Data
Dec. 10, 2014 (DE) 10 2014 225 472.8

(51) Int. Cl. C11D 3/386; C12N 9/26; C11D 11/00

(52) U.S. Cl. CPC C11D 3/38618; C11D 11/0023; C12N 9/2414; C12Y 302/01001

(57) ABSTRACT

Disclosed is a liquid cleaning agent for hard surfaces, in particular for washing dishes by hand, containing an a amylase which is at least 89%, with increasing preference at least 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5% and up to 100% identical to the sequence indicated in SEQ ID NO: 1 along the entire length thereof and which has deletions in one or more of positions 180, 181, 182, 183 and 184 as numbered according to SEQ ID NO: 1.

### FIELD OF THE INVENTION

The present invention generally relates to a liquid cleaning agent, in particular an aqueous cleaning agent for hard surfaces. The cleaning agent is preferably used for washing dishes by hand.

### BACKGROUND OF THE INVENTION

Conventional liquid cleaning agents contain surfactants for removing dirt and stains. As a rule, in this connection combinations of several surfactants are used, in particular from the group of anionic, non-ionic, cationic and amphoteric surfactants. These surfactants alone are frequently incapable of removing dirt and stains adequately, with the result that further auxiliaries are used in modern washing or cleaning agents. These further auxiliaries include enzymes of different types such as proteases, amylases, cellulases, mannanases or pectate lyases. Further classes of enzyme are known to a person skilled in the art. Amylases take on a particular importance here, due to their effectiveness against dried starch, because easier cleaning at lower mechanical outlay is achieved through their use.

However, the performance of amylases used thus far in hand dishwashing detergents depends greatly on temperature, with the result that the advantages of these active ingredients, which manifest at washing temperatures of approximately 40° C., are considerably diminished at lower temperatures, and hardly any effect can be recognized at 20° C. On the other hand, however, for reasons of sustainability and energy efficiency, it is desirable that there is an effective soaking effect against starch also at low temperatures.

Therefore, the object of the invention is to provide a liquid cleaning agent, in particular for hard surfaces, which has a good cleaning capacity vis-à-vis starch-containing and dried stains, even at low temperatures.

Furthermore, other desirable features and characteristics of the present invention will become apparent from the subsequent detailed description of the invention and the appended claims, taken in conjunction with this background of the invention.

### BRIEF SUMMARY OF THE INVENTION

Therefore, the subject matter of the present invention is a liquid cleaning agent containing an a-Amylase, which is at least 89%, and with increasing preference at least 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5% and up to 100% identical to the sequence indicated in SEQ ID NO. 1 along the entire length thereof, and which has deletions in one or more of positions 180, 181, 182, 183 and 184 as numbered according to SEQ ID NO. 1.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Surprisingly, it has been found that this object is achieved by a liquid cleaning agent containing an a-Amylase, which is at least 89%, and with increasing preference at least 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5% and up to 100% identical to the sequence indicated in SEQ ID NO. 1 along the entire length thereof, and which has deletions in one or more of positions 180, 181, 182, 183 and 184 as numbered according to SEQ ID NO. 1. With such a cleaning agent, very good cleaning results can already be achieved, in particular in removing starch, also at temperatures clearly below 40° C.

A deletion of two positions, selected from 180+181, 181+182, 182+183 and 183+184, is particularly preferred, while deletions at positions 183+184 as numbered according to SEQ ID NO. 1 are quite particularly preferred, and in particular deletions H183*+G184* are preferred.

Preferably, the α-Amylase according to the invention furthermore has an amino acid substitution at one or more of positions 405, 421, 422 and 428 as numbered according to SEQ ID NO. 1. Substitutions 1405L; A421H, A422P and A428T are particularly preferred.

In a particularly preferred embodiment, the a-Amylase according to the invention has deletions H183*+G184* and additionally substitutions 1405L, A421H, A422P and A428T as numbered according to SEQ ID NO. 1.

Amino acid positions which, within the scope of the present invention, are indicated as containing the phrase “numbered according to SEQ ID NO. 1,” are understood as follows: the further amino acid positions are defined by an alignment of the amino acid sequence of an amylase according to the invention with the amino acid sequence as indicated in SEQ ID NO. 1. Furthermore, the positions are allocated according to the mature protein. This allocation is in particular also to be applied if the amino acid sequence of a protein according to the invention comprises a higher number of amino acid residues than the amylase in SEQ ID NO. 1. Proceeding from the named positions in the amino acid sequence, the change positions in an amylase according to the invention are those which are allocated precisely these positions in an alignment.

A further subject matter of the present invention is the use of a liquid cleaning agent according to the invention for washing dishes by hand. Yet a further subject matter of the present invention is a method for cleaning hard surfaces, in particular for washing dishes by hand, comprising the use of a liquid cleaning agent according to the invention.

Within the scope of the present invention, liquid cleaning agents with an α-Amylase content of from 0.05 to 1.0 wt.-%, preferably of from 0.1 to 0.4 wt.-%, are particularly preferred.

By liquid cleaning agents are meant, within the scope of the present invention, those which are flowable under normal service conditions, and the viscosities of which may vary within a broad range. These liquid preparations also include gel or pasty agents which can optionally contain additional thickeners known from the prior art. In a further preferred embodiment of the invention, the liquid agents are water-based. In particular, the cleaning agents according to the invention are hand dishwashing detergents.

In the context of the present invention, a hand dishwashing detergent is a liquid washing or cleaning agent which is particularly adapted to use for washing dishes by hand. Accordingly, hand dishwashing detergents are particularly suitable for removing dirt from hard surfaces, have good foaming performance and also a particular skin compatibility.

Advantageously, the cleaning agent according to the present invention is designed preferably as a hand dishwashing detergent which has a foaming performance of at least 250 mL, measured according to DIN method 53 902, part 2 (Ross-Miles test), preferably of at least 300 mL. This advantageous foaming behavior can typically be traced back to the hand dishwashing detergent preferably containing at least 5 wt.-% of an anionic surfactant relative to the whole hand dishwashing detergent. The total content of surfactant may, however, be clearly higher (see below).

In order on the one hand to guarantee good skin compatibility and on the other hand to make possible an optimum effectiveness of the enzymes contained, the pH of the hand dishwashing detergent is preferably in the range of from (in each case inclusive) 6.0 to 9.0. What is particularly preferred is pH values between pH 7.0 and pH 8.0 (in each case inclusive).

As further constituents of conventional surfactants, the cleaning agents according to the invention contain above all anionic surfactants, non-ionic surfactants, amphoteric surfactants, betaines and optionally cationic surfactants. The total quantity of surfactants in the agents according to the invention may vary within a broad range, and for example be 3 to 70 wt.-%, preferably 5 to 50 wt.-% and in particular 10 to 40 wt.-%.

The anionic surfactants are conventionally used as alkali metal salt, alkaline-earth metal salt and/or mono-, di- or trialkanol ammonium salt and/or, however, also in the form of the corresponding alkali metal hydroxide, alkaline-earth metal hydroxide and/or mono-, di- or trialkanol amine in situ, to the neutralizing corresponding acid. Potassium and in particular sodium are preferred here as alkali metals, calcium and in particular magnesium as alkaline-earth metals, and mono-, di- or triethanol amine as alkanol amines. Sodium salts are particularly preferred.

Preferably, anionic surfactants used in particular for washing dishes by hand include, above all, alkyl ether sulfates and alkyl sulfonates.

Alkyl ether sulfates (fatty alcohol ether sulfates, INCI: alkyl ether sulfates) are products of sulfated reactions on alkoxylated alcohols. Generally, by alkoxylated alcohols, a person skilled in the art understands the reaction products of alkylene oxide, preferably ethylene oxide, with alcohols, within the meaning of the present invention preferably with longer-chained alcohols, i.e. with aliphatic straight-chained or single or multiple branched, acyclic or cyclic, saturated or single or multiple unsaturated, preferably straight-chained, acyclic, saturated, alcohols with 6 to 22, preferably 8 to 18, in particular 10 to 16 and particularly preferably 12 to 14 carbon atoms. As a rule, from n mols ethylene oxide and one mol alcohol, the result is a complex mixture of products of addition of different degrees of ethoxylation (n=1 to 30, preferably 1 to 20, in particular 1 to 10, particularly preferably 2 to 4), depending on the reaction conditions. A further embodiment of the alkoxylation consists of the use of mixtures of alkylene oxides, preferably of the mixture of ethylene oxide and propylene oxide. Quite particularly preferred within the meaning of the present invention are low-ethoxylated fatty alcohols with 1 to 4 ethylene oxide units (EO), in particular 1 to 2 EO, for example 2 EO, such as Na—C12-14 fatty alcohol+2EO sulfate. In a preferred embodiment, the agent according to the invention contains at least one alkyl ether sulfate.

The alkyl sulfonates (INCI sulfonic acids) conventionally have an aliphatic straight-chained or single or multiple branched, acyclic or cyclic, saturated or single or multiple unsaturated, preferably branched, acyclic, saturated, alkyl residue with 6 to 22, preferably 9 to 20, in particular 11 to 18 and particularly preferably 14 to 17 carbon atoms.

Suitable alkyl sulfonates are accordingly the saturated alkane sulfonates, the unsaturated olefin sulfonates and the ether sulfonates—deriving formally from the alkoxylated alcohols also forming the basis of the alkyl ether sulfates—in which a distinction is drawn between terminal ether sulfonates (n-ether sulfonates) with sulfonate function bonded to the polyether chain and internal ether sulfonates (i-ether sulfonates) with sulfonate function bonded to the alkyl residue.

According to the invention, the alkane sulfonates, in particular alkane sulfonates with a branched, preferably secondary, alkyl residue, for example the secondary alkane sulfonate sec. Na—C₁₃₋₁₇ alkane sec. sulfonate (INCI sodium C14-17 alkyl sec sulfonate) are particularly preferred.

Further possible anionic surfactants which can be used are known to a person skilled in the art from the relevant prior art in respect of washing or cleaning agents. These include, in particular, aliphatic sulfates such as fatty alcohol sulfates, monoglyceride sulfates as well as ester sulfonates (sulfo fatty acid esters), lignin sulfonates, alkylbenzene sulfonates, fatty acid cyanamides, anionic sulfosuccinic acid surfactants, fatty acid isethionates, acylamino alkane sulfonates (fatty acid taurides), fatty acid sarcosinates, ether carboxylic acids and alkyl(ether) phosphates.

Suitable further anionic surfactants are also anionic gemini surfactants with a diphenyl oxide framework, 2 sulfonate groups and an alkyl residue at one or both benzene rings according to the formula ⁻O₃S(C₆H₃R)O(C₆H₃R′)SO₃, in which R stands for an alkyl residue with for example 6, 10, 12 or 16 carbon atoms and R′ stands for R or H (Dowfax® Dry Hydrotrope Powder with C₁₆ alkyl residue(s); INCI sodium hexyldiphenyl ether sulfonate, disodium decyl phenyl ether disulfonate, disodium lauryl phenyl ether disulfonate, disodium cetyl phenyl ether disulfonate).

Particularly preferred further anionic surfactants are the anionic sulfosuccinic acid surfactants sulfosuccinate, sulfosuccinamate and sulfosuccinamide, in particular sulfosuccinate and sulfosuccinamate, with sulfosuccinate being extremely preferred. The sulfosuccinates are the salts of mono- and diesters of sulfosuccinic acid HOOCCH(SO₃H)CH₂COOH, while sulfosuccinamates are understood to mean the salts of the monoamides of sulfosuccinic acid and sulfosuccinamides are understood to mean the salts of the diamides of sulfosuccinic acid. The salts are preferably alkali metal salts, ammonium salts as well as mono-, di- or trialkanol ammonium salts, for example mono-, di- or triethanol ammonium salts, in particular lithium, sodium, potassium or ammonium salts, particularly preferably sodium or ammonium salts, extremely preferably sodium salts.

In the sulfosuccinates, one or both carboxyl groups of the sulfosuccinic acid are esterified, preferably with one or two identical or different, unbranched or branched, saturated or unsaturated, acyclic or cyclic, optionally alkoxylated alcohols with 4 to 22, preferably 6 to 20, in particular 8 to 18, particularly preferably 10 to 16, and extremely preferably 12 to 14 carbon atoms. Particularly preferably, the esters are unbranched and/or saturated and/or acyclic and/or alkoxylated alcohols, in particular unbranched, saturated fatty alcohols and/or unbranched, saturated fatty alcohols, alkoxylated with ethylene and/or propylene oxide, preferably ethylene oxide, with a degree of alkoxylation of from 1 to 20, preferably 1 to 15, in particular 1 to 10, particularly preferably 1 to 6, and extremely preferably 1 to 4. Within the scope of the invention, monoesters are preferred to diesters. A particularly preferred sulfosuccinate is sulfosuccinic acid lauryl polyglycol ester disodium salt (Lauryl-EO-sulfosuccinate, Di-Na salt; INCI disodium laureth sulfosuccinate), which for example can be obtained commercially as Tego® sulfosuccinate F 30 (Goldschmidt) with a sulfosuccinate content of 30 wt.-%.

In the sulfosuccinamates or sulfosuccinamides, one or both carboxyl groups of the sulfosuccinic acid form(s) a carboxylic acid amide, preferably with a primary or secondary amine, which has one or two identical or different, unbranched or branched, saturated or unsaturated, acyclic or cyclic, optionally alkoxylated alkyl residues with 4 to 22, preferably 6 to 20, in particular 8 to 18, particularly preferably 10 to 16, and extremely preferably 12 to 14 carbon atoms. Unbranched and/or saturated and/or acyclic alkyl residues, in particular unbranched, saturated fatty alcohol residues, are particularly preferred.

Moreover, the following sulfosuccinates and sulfosuccinamates denoted according to INCI, which are described in more detail in the International Cosmetic Ingredient Dictionary and Handbook, are suitable: ammonium dinonyl sulfosuccinate, ammonium lauryl sulfosuccinate, diammonium dimethicone copolyol sulfosuccinate, diammonium lauramido-MEA sulfosuccinate, diammonium lauryl sulfosuccinate, diammonium oleamido PEG-2 sulfosuccinate, diamyl sodium sulfosuccinate, dicapryl sodium sulfosuccinate, dicyclohexyl sodium sulfosuccinate, diheptyl sodium sulfosuccinate, dihexyl sodium sulfosuccinate, diisobutyl sodium sulfosuccinate, dioctyl sodium sulfosuccinate, disodium cetearyl sulfosuccinate, disodium cocamido MEA-sulfosuccinate, disodium cocamido MIPA-sulfosuccinate, disodium cocamido PEG-3 sulfosuccinate, disodium coco-glucoside sulfosuccinate, disodium cocoyl butyl gluceth-10 sulfosuccinate, disodium C12-15 pareth sulfosuccinate, disodium deceth-5 sulfosuccinate, disodium deceth-6 sulfosuccinate, disodium dihydroxyethyl sulfosuccinylundecylenate, disodium dimethicone copolyol sulfosuccinate, disodium hydrogenated cottonseed glyceride sulfosuccinate, disodium isodecyl sulfosuccinate, disodium isostearamido MEA-sulfosuccinate, disodium isostearamido MIPA-sulfosuccinate, disodium isostearyl sulfosuccinate, disodium laneth-5 sulfosuccinate, disodium lauramido MEA-sulfosuccinate, disodium lauramido PEG-2 sulfosuccinate, disodium lauramido PEG-5 sulfosuccinate, disodium laureth-6 sulfosuccinate, disodium laureth-9 sulfosuccinate, disodium laureth-12 sulfosuccinate, disodium lauryl sulfosuccinate, disodium myristamido MEA-sulfosuccinate, disodium nonoxynol-10 sulfosuccinate, disodium oleamido MEA-sulfosuccinate, disodium oleamido MIPA-sulfosuccinate, disodium oleamido PEG-2 sulfosuccinate, disodium oleth-3 sulfosuccinate, disodium oleyl sulfosuccinate, disodium palmitamido PEG-2 sulfosuccinate, disodium palmitoleamido PEG-2 sulfosuccinate, disodium PEG-4 cocamido MIPA-sulfosuccinate, disodium PEG-5 laurylcitrate sulfosuccinate, disodium PEG-8 palm glycerides sulfosuccinate, disodium ricinoleamido MEA-sulfosuccinate, disodium sitostereth-14 sulfosuccinate, disodium stearamido MEA-sulfosuccinate, disodium stearyl sulfosuccinamate, disodium stearyl sulfosuccinate, disodium tallamido MEA-sulfosuccinate, disodium tallowamido MEA-sulfosuccinate, disodium tallow sulfosuccinamate, disodium tridecylsulfosuccinate, disodium undecylenamido MEA-sulfosuccinate, disodium undecylenamido PEG-2 sulfosuccinate, disodium wheat germamido MEA-sulfosuccinate, disodium wheat germamido PEG-2 sulfosuccinate, di-TEA-oleamido PEG-2 sulfosuccinate, ditridecyl sodium sulfosuccinate, sodium bisglycol ricinosulfosuccinate, sodium/MEA laureth-2 sulfosuccinate and tetrasodium dicarboxyethyl stearyl sulfosuccinamate. Another suitable sulfosuccinamate is disodium C₁₆₋₁₈ alkoxy propylene sulfosuccinamate.

Preferred anionic sulfosuccinic acid surfactants are imidosuccinate, mono-Na sulfosuccinic acid diisobutylester (Monawet® MB 45), mono-Na sulfosuccinic acid dioctylester (Monawet® MO-84 R2W, Rewopol® SB DO 75), mono-Na sulfosuccinic acid ditridecylester (Monawet® MT 70), fatty alcohol polyglycol sulfosuccinate-Na—NH₄ salt (sulfosuccinate S-2), di-Na-sulfosuccinic acid mono-C_{12/14}-3EO ester (Texapon® SB-3), sodium sulfosuccinic acid diisooctylester (Texin® DOS 75) and di-Na-sulfosuccinic acid mono-C_{12/18} ester (Texin® 128-P), in particular the mono-Na sulfosuccinic acid dioctylester which interacts synergistically with the ternary surfactant combination according to the invention in respect of the draining and/or drying characteristics.

In a particular embodiment, the agent according to the invention contains, as anionic sulfosuccinic acid surfactants, one or more sulfosuccinates, sulfosuccinamates and/or sulfosuccinamides, preferably sulfosuccinates and/or sulfosuccinamates, in particular sulfosuccinates, in a quantity of conventionally 0.001 to 5 wt.-%, preferably 0.01 to 4 wt.-%, in particular 0.1 to 3 wt.-%, particularly preferably 0.2 to 2 wt.-%, extremely preferably 0.5 to 1.5 wt.-%, for example 1 wt.-%.

In a preferred embodiment of the present invention, the proportion of anionic surfactant is 5 to 20 wt.-%, particularly preferably 8 to 16 wt.-%, relative to the total weight of the cleaning agent, in particular of the hand dishwashing detergent. In a particularly preferred embodiment, the cleaning agent according to the invention contains 10 wt.-% anionic surfactant. The anionic surfactant is preferably a fatty alcohol ether sulfate. Preferred fatty alcohol ether sulfates are described above.

The amphoteric surfactants and zwitterionic surfactants which can be used according to the invention include alkylamidoalkylamines, alkyl-substituted amino acids, acylated amino acids or biosurfactants, with betaines within the scope of the teaching according to the invention being preferred.

Suitable betaines which are used above all in hand dishwashing detergents are alkylbetaines, alkylamidobetaines, imidazoliniumbetaines, sulfobetaines (INCI sultaines) as well as phosphobetaines, and preferably satisfy formula I,

R¹—[CO—X—(CH₂)ₙ]ₓ—N⁺(R²)(R^{3)—(CH}₂)ₘ—[CH(OH)—CH₂]_{y}—Y⁻ (I)
in which R¹ is a saturated or unsaturated C₆₋₂₂ alkyl residue, preferably C₈₋₁₈ alkyl residue, in particular a saturated C₁₀₋₁₆ alkyl residue, for example a saturated C₁₂₋₁₄ alkyl residue,
   X is NH, NR⁴ with the C₁₋₄ alkyl residue R⁴, O or S,
   n is a number from 1 to 10, preferably 2 to 5, in particular 3,
   x is 0 or 1, preferably 1,
   R², R³ independently of one another is a C₁₋₄ alkyl residue, optionally hydroxysubstituted such as e.g. a hydroxyethyl residue, but in particular a methyl residue,
   m is a number from 1 to 4, in particular 1, 2 or 3,
   y is 0 or 1 and
   Y is COO, SO_{3,} OPO(OR⁵)O or P(O)(OR⁵)O, wherein R⁵ is a hydrogen atom H or a C₁₋₄ alkyl residue.

The alkyl and alkylamidobetaines, betaines of formula I with a carboxylate group (Y⁻=COO⁻), are also called carbobetaines.

Preferred betaines are the alkylbetaines of formula (la), the alkylamidobetaines of formula (Ib), the sulfobetaines of formula (Ic) and the amidosulfobetaines of formula (Id),

R¹—N⁺(CH₃)₂—CH₂COO⁻ (la)

R¹—CO—NH—(CH₂)₃—N⁺(CH₃)₂—CH₂COO⁻ (Ib)

R¹—N⁺(CH₃)₂—CH₂CH(OH)CH₂SO₃⁻ (Ic)

R₁—CO—NH—(CH₂)₃—N⁺(CH₃)₂—CH₂CH(OH)CH₂SO₃⁻ (Id)

in which R¹ has the same meaning as in formula I.

Particularly preferred betaines are carbobetaines, in particular the carbobetaines of formula (la) and (Ib), extremely preferably the alkylamidobetaines of formula (Ib).

Examples of suitable betaines and sulfobetaines are the following compounds named according to INCL almondamidopropyl betaine, apricotamidopropyl betaine, avocadamidopropyl betaine, babassuamidopropyl betaine, behenamidopropyl betaine, behenyl betaine, betaine, canolamidopropyl betaine, capryl/capramidopropyl betaine, carnitine, cetyl betaine, cocamidoethyl betaine, cocamidopropyl betaine, cocamidopropyl hydroxysultaine, coco-betaine, coco-hydroxy-sultaine, coco/oleamidopropyl betaine, coco-sultaine, decyl betaine, dihydro-xyethyl oleyl glycinates, dihydroxyethyl soy glycinates, dihydroxyethyl stearyl glycinates, dihydroxyethyl tallow glycinates, dimethicone propyl PG-betaine, erucamidopropyl hydroxysultaine, hydrogenated tallow betaine, isostearamidopropyl betaine, lauramidopropyl betaine, lauryl betaine, lauryl hydroxysultaine, lauryl sultaine, milkamidopropyl betaine, minkamidopropyl betaine, myristamidopropyl betaine, myristyl betaine, oleamidopropyl betaine, oleamidopropyl hydroxysultaine, oleyl betaine, olivamidopropyl betaine, palmamidopropyl betaine, palmitamidopropyl betaine, palmitoyl carnitine, palm kernelamidopropyl betaine, polytetrafluoroethylene acetoxypropyl betaine, ricinoleamidopropyl betaine, sesamidopropyl betaine, soyamidopropyl betaine, stearamidopropyl betaine, stearyl betaine, tallowamidopropyl betaine, tallowamidopropyl hydroxysultaine, tallow betaine, tallow dihydroxyethyl betaine, undecylenamidopropyl betaine and wheat germamidopropyl betaine. A preferred betaine is for example cocamidopropyl betaine.

The alkylamido alkylamines are amphoteric surfactants of formula (III),

R⁹—CO—NR¹⁰—(CH₂)ᵢ—N(R¹¹)—(CH₂CH₂O)ⱼ—(CH₂)ₖ—[CH(OH)ₗ—CH₂—Z—OM (III)
in whichR⁹ is a saturated or unsaturated C₆₋₂₂ alkyl residue, preferably C₈₋₁₈ alkyl residue, in particular a saturated C₁₀₋₁₆ alkyl residue, for example a saturated C₁₂₋₁₄ alkyl residue,
   R¹⁰ is a hydrogen atom H or a C₁₋₄ alkyl residue, preferably H,
   i is a number from 1 to 10, preferably 2 to 5, in particular 2 or 3,
   R¹¹ is a hydrogen atom H or CH₂COOM (for M, see below),
   j is a number from 1 to 4, preferably 1 or 2, in particular 1,
   k is a number from 0 to 4, preferably 0 or 1,
   l is 0 or 1, wherein k=1 if I=1,
   Z is CO, SO_{2,} OPO(OR¹²) or P(O)(OR¹²), wherein ^{R12} is a C₁₋₄ alkyl residue or M (see below), and
   M is a hydrogen, an alkali metal, an alkaline-earth metal or a protonated alkanolamine, e.g. protonated mono-, di- or triethanolamine.

Preferred representatives satisfy formulae IIIa to IIId,

R⁹—CO—NH—(CH₂)₂—N(R¹¹)—CH₂CH₂O—CH₂—COOM (IIIa)

R⁹—CO—NH—(CH₂)₂—N(R¹¹)—CH₂CH₂O—CH₂CH₂—COOM (IIIb)

R⁹—CO—NH—(CH₂)₂—N(R¹¹)—CH₂CH₂O—CH₂CH(OH)CH₂—SO₃M (IIIc)

R⁹—CO—NH—(CH₂)₂—N(R¹¹)—CH₂CH₂O—CH₂CH(OH)CH₂—OPO₃HM (IIId)
in which R¹¹ and M have the same meaning as in formula (III).

Exemplary alkylamidoalkylamines are the following compounds named according to INCI: cocoamphodipropionic acid, cocobetainamido amphopropionate, DEA-cocoamphodipropionate, disodium caproamphodiacetate, disodium caproamphodipropionate, disodium capryloamphodiacetate, disodium capryloamphodipropionate, disodium cocoamphocarboxyethylhydroxypropylsulfonate, disodium cocoamphodiacetate, disodium cocoamphodipropionate, disodium isostearoamphodiacetate, disodium isostearoamphodipropionate, disodium laureth-5 carboxyamphodiacetate, disodium lauroamphodiacetate, disodium lauroamphodipropionate, disodium oleoamphodipropionate, disodium PPG-2-isodeceth-7 carboxyamphodiacetate, disodium stearoamphodiacetate, disodium tallowamphodiacetate, disodium wheatgermamphodiacetate, lauroamphodipropionic acid, quaternium-85, sodium caproamphoacetate, sodium caproamphohydroxypropylsulfonate, sodium caproamphopropionate, sodium capryloamphoacetate, sodium capryloamphohydroxypropylsulfonate, sodium capryloamphopropionate, sodium cocoamphoacetate, sodium cocoamphohydroxypropylsulfonate, sodium cocoamphopropionate, sodium cornamphopropionate, sodium isostearoamphoacetate, sodium isostearoamphopropionate, sodium lauroamphoacetate, sodium lauroamphohydroxypropylsulfonate, sodium lauroampho PG-acetate phosphate, sodium lauroamphopropionate, sodium myristoamphacetate, sodium oleoamphoacetate, sodium oleoamphohydroxypropylsulfonate, sodium oleoamphopropionate, sodium ricinoleoamphoacetate, sodium stearoamphoacetate, sodium stearoamphohydroxypropylsulfonate, sodium stearoamphopropionate, sodium tallamphopropionate, sodium tallowamphoacetate, sodium undecylenoamphoacetate, sodium undecylenoamphopropionate, sodium wheat germamphoacetate and trisodium lauroampho PG-acetate chloride phosphate.

Alkyl-substituted amino acids preferred according to the invention are monoalkyl-substituted amino acids according to formula (IV),

R¹³—NH—CH(R¹⁴)—(CH₂)ᵤ—COOM′ (IV)
in which R¹³ is a saturated or unsaturated C₆₋₂₂ alkyl residue, preferably C₈₋₁₈ alkyl residue, in particular a saturated C₁₀₋₁₆ alkyl residue, for example a saturated C₁₂₋₁₄ alkyl residue,
   R₁₄ is a hydrogen atom H or a C₁₋₄ alkyl residue, preferably H,
   u is a number from 0 to 4, preferably 0 or 1, in particular 1, and
   M′ is a hydrogen, an alkali metal, an alkaline-earth metal or a protonated alkanolamine, e.g. protonated mono-, di- or triethanolamine,

Alkyl-substituted imino acids according to formula (V),

R¹⁵—N—[(CH₂)ᵥ—COOM″]₂ (V)
in whichR¹⁵ is a saturated or unsaturated C₆₋₂₂ alkyl residue, preferably C₈₋₁₈ alkyl residue, in particular a saturated C₁₀₋₁₆ alkyl residue, for example a saturated C₁₂₋₁₄ alkyl residue,
   v is a number from 1 to 5, preferably 2 or 3, in particular 2, and
   M″ is a hydrogen, an alkali metal, an alkaline-earth metal or a protonated alkanolamine, e.g. protonated mono-, di- or triethanolamine, wherein M″ in the two carboxy groups can have the same or two different meanings, e.g. hydrogen and sodium, or can both be sodium,

and mono- or dialkyl-substituted natural amino acids according to formula (VI),

R¹⁶—N(R¹⁷)—CH(R¹⁸)—COOM′″ (VI)
in whichR¹⁶ is a saturated or unsaturated C₆₋₂₂ alkyl residue, preferably C₈₋₁₈ alkyl residue, in particular a saturated C₁₀₋₁₆ alkyl residue, for example a saturated C₂₋₁₄ alkyl residue,
   R¹⁷ is a hydrogen atom or a C₁₋₄ alkyl residue, optionally hydroxy- or amine-substituted, e.g. a methyl-, ethyl-, hydroxyethyl- or aminopropyl residue,
   R¹⁸ is the radical of one of the 20 natural α-amino acids H₂NCH(R¹⁸)COOH, and
   M′″ is a hydrogen, an alkali metal, an alkaline-earth metal or a protonated alkanolamine, e.g. protonated mono-, di- or triethanolamine.

Particularly preferred alkyl-substituted amino acids are the aminopropionates according to formula (IVa),

R¹³—NH—CH₂CH₂COOM′ (IVa)
in which R¹³ and M′ have the same meaning as in formula (IV).

Exemplary alkyl-substituted amino acids are the following compounds named according to INCI: aminopropyl laurylglutamine, cocaminobutyric acid, cocaminopropionic acid, DEA-lauraminopropionate, disodium cocaminopropyl iminodiacetate, disodium dicarboxyethyl cocopropylenediamine, disodium lauriminodipropionate, disodium steariminodipropionate, disodium tallowiminodipropionate, lauraminopropionic acid, lauryl aminopropylglycine, lauryl diethylenediaminoglycine, myristaminopropionic acid, sodium C12-15 alkoxypropyl iminodipropionate, sodium cocaminopropionate, sodium lauraminopropionate, sodium lauriminodipropionate, sodium lauroyl methylaminopropionate, TEA-lauraminopropionate and TEA-myristaminopropionate.

Acylated amino acids are amino acids, in particular the 20 natural α-amino acids which have, at the amino nitrogen atom, the acyl residue R¹⁹CO of a saturated or unsaturated fatty acid R¹⁹COOH, wherein R¹⁹ is a saturated or unsaturated C₆₋₂₂ alkyl residue, preferably C₈₋₁₈ alkyl residue, in particular a saturated C₁₀₋₁₆ alkyl residue, for example a saturated C₁₂₋₁₄ alkyl residue. The acylated amino acids can also be used as an alkali metal salt, alkaline-earth metal salt or alkanolammonium salt, e.g. mono-, di- or triethanolammonium salt. Exemplary acylated amino acids are the acyl derivatives summarized according to the INCI under amino acids, e.g. sodium cocoyl glutamate, lauroyl glutamic acid, capryloyl glycine or myristoyl methylalanine.

In a particular embodiment of the invention, a combination of two or more different amphoteric surfactants, in particular a binary amphoteric surfactant combination, is used.

The amphoteric surfactant combination contains preferably at least one betaine, in particular at least one alkylamidobetaine, particularly preferably cocamidopropyl betaine.

Furthermore, the amphoteric surfactant combination contains preferably at least one amphoteric surfactant from the group comprising sodium carboxyethyl cocophosphoethyl imidazoline (Phosphoteric® TC-6), C_{8/10} amidopropyl betaine (INCI capryl/capramidopropyl betaine; Tego° betaine 810), N-2-hydroxyethyl-N-carboxymethyl-fatty acid amidoethylamine-Na (Rewoteric® AMV) and N-capryl/caprinamidoethyl-N-ethylether-propionate-Na (Rewoteric® AMVSF) as well as betaine 3-(3-cocamidopropyl)-dimethylammonium-2-hydroxypropane sulfonate (INCI sultaine; Rewoteric® AM CAS) and the alkylamido alkylamine N-[N′(N″-2-hydroxyethyl-N″-carboxyethylaminoethyl)-acetic acid amido]-N,N-dimethyl-N-cocoammonium betaine (Rewoteric® QAM 50), in particular together with cocamidopropyl betaine.

In a preferred embodiment, the agent according to the invention contains one or more amphoteric surfactants, in particular betaines, in a quantity of from 0.5 to 15 wt.-%, preferably 1 to 10 wt.-%, in particular 2 to 8 wt.-%. In a quite particularly preferred embodiment, the agent contains 4 wt.-% amphoteric surfactants (in particular betaine). Preferred betaines have been described above. A quite particularly preferred betaine is cocamidopropyl betaine.

In a preferred embodiment, the agent according to the invention contains a surfactant combination made of at least one anionic surfactant and at least one amphoteric surfactant. These surfactants are present preferably in a mass ratio of from 10:1 to 1:5, preferably of from 5:1 to 2:1, in particular preferably of from 4:1. Preferably, the at least one anionic surfactant comprises at least one alkyl ether sulfate and the at least one amphoteric surfactant comprises at least one betaine surfactant.

Preferably alkoxylated, advantageously ethoxylated, in particular primary alcohols with preferably 8 to 18 C atoms and on average 1 to 12 mols ethylene oxide (EO) per mol alcohol are used as non-ionic surfactants, in which the alcohol residue can be linear or preferably methyl-branched in 2 position or can contain linear and methyl-branched residues in the mixture, as are conventionally present in oxo alcohol residues. However, in particular alcohol ethoxylates with linear residues made of alcohols of native origin with 12 to 18 C atoms, for example made of coco-, palm-, tallow fat or oleyl alcohol, and on average 2 to 8 EO per mol alcohol are preferred. The preferred ethoxylated alcohols include for example C₁₂₋₁₄ alcohols with 3 EO, 4 EO or 7 EO, C₉₋₁₁ alcohol with 7 EO, C₁₃₋₁₅ alcohols with 3 EO, 5 EO, 7 EO or 8 EO, C₁₂₋₁₈ alcohols with 3 EO, 5 EO or 7 EO and mixtures thereof, such as mixtures of C₁₂₋₁₄ alcohol with 3 EO and C₁₂₋₁₈ alcohol with 7 EO. The indicated degrees of ethoxylation represent statistical averages which can be an integer or a fractional number for a special product. Preferred alcohol ethoxylates have a concentrated homolog distribution (narrow range ethoxylates, NRE). In addition to these non-ionic surfactants, fatty alcohols with more than 12 EO can also be used. Examples of this are tallow fatty alcohol with 14 EO, 25 EO, 30 EO or 40 EO. Also non-ionic surfactants, which contain EO and PO groups together in the molecule can be used according to the invention. Particularly preferably, the hand dishwashing detergent contains a C₁₂₋₁₈ fatty alcohol with 7 EO or a C₁₃₋₁₅ oxoalcohol with 7 EO as non-ionic surfactant.

In a further preferred embodiment of the liquid cleaning agent, in particular of the hand dishwashing detergent, it is preferred as a further surfactant that a non-ionic surfactant, preferably a fatty alcohol polyglycol ether, be contained, preferably in quantities of from 0.00 1 to 10 wt.-%, particularly preferably 0.01 to 6 wt.-%, further preferably 0.1 to 4 wt.-%, in particular 0.2 to 2 wt.-%., in each case relative to the whole hand dishwashing detergent.

In combination with an amine oxide, these non-ionic surfactants have a good cleaning performance on greasy hard surfaces, such as for example dishes.

Non-ionic surfactants within the scope of the invention are alkoxylates, but also alkylphenol polyglycol ether, terminal group-capped polyglycol ether, mixed ether and hydroxy mixed ether and fatty acid polyglycol ester. Likewise suitable are block polymers of ethylene oxide and propylene oxide as well as fatty acid alkanolamides and fatty acid polyglycol ethers. Important classes of non-ionic surfactants according to the invention are furthermore amine oxides and sugar surfactants, in particular alkyl polyglucosides.

The amine oxides suitable according to the invention include alkylamine oxides, in particular alkyl dimethyl amine oxide, alkyl amidoamine oxide and alkoxy alkylamine oxide. Preferred amine oxides satisfy formula II,

R⁶R⁷R⁸N⁺—O⁻ (II)

R⁶—[CO—NH—(CH₂)_{w}]_{z}—N⁺(R⁷)(R⁸)—O⁻ (III)
in whichR⁶ is a saturated or unsaturated C₆₋₂₂ alkyl residue, preferably C₈₋₁₈ alkyl residue, in particular a saturated C₁₀₋₁₆ alkyl residue, for example a saturated C₁₂₋₁₄ alkyl residue, which is bonded in the alkylamido amine oxides via a carbonyl amidoalkylene group —CO—NH—(CH₂)_{z}—and in the alkoxyalkyl amine oxides via an oxa-alkylene group —O—(CH₂) to the nitrogen atom N, wherein z in each case stands for a number from 1 to 10, preferably 2 to 5, in particular 3,
   R⁷, R⁸ independently of one another is a C₁₋₄ alkyl residue, optionally hydroxy-substituted such as e.g. a hydroxyethyl residue, in particular a methyl residue.

Examples of suitable amine oxides are the following compounds named according to INCI: almondamidopropylamine oxide, babassuamidopropylamine oxide, behenamine oxide, cocamidopropyl amine oxide, cocamidopropylamine oxide, cocamine oxide, coco-morpholine oxide, decylamine oxide, decyltetradecylamine oxide, diaminopyrimidine oxide, dihydroxyethyl C₈₋₁₀ alkoxypropylamine oxide, dihydroxyethyl C₉₋₁₁ alkoxypropylamine oxide, dihydroxyethyl C₁₂₋₁₅ alkoxypropylamine oxide, dihydroxyethyl cocamine oxide, dihydroxyethyl lauramine oxide, dihydroxyethyl stearamine oxide, dihydroxyethyl tallowamine oxide, hydrogenated palm kernel amine oxide, hydrogenated tallowamine oxide, hydroxyethyl hydroxypropyl C₁₂₋₁₅ alkoxypropylamine oxide, isostearamidopropylamine oxide, isostearamidopropyl morpholine oxide, lauramidopropylamine oxide, lauramine oxide, methyl morpholine oxide, milkamidopropylamine oxide, minkamidopropylamine oxide, myristamidopropylamine oxide, myristamine oxide, myristyl/cetyl amine oxide, oleamidopropylamine oxide, oleamine oxide, olivamidopropylamine oxide, palmitamidopropylamine oxide, palmitamine oxide, PEG-3 lauramine oxide, potassium dihydroxyethyl cocamine oxide phosphate, potassium trisphosphonomethylamine oxide, sesamidopropylamine oxide, soyamidopropylamine oxide, stearamidopropylamine oxide, stearamine oxide, tallowamidopropylamine oxide, tallowamine oxide, undecylenamidopropylamine oxide and wheat germamidopropylamine oxide. Preferred amine oxides are for example cocamidopropylamine oxide but also N-cocoalkyl-N,N-dimethyl amine oxide, N-tallowalkyl-N, N-dihydroxyethylamine oxide, myristylcetyl dimethyl amine oxide or lauryl dimethyl amine oxide.

The amine oxide content in the agent according to the invention is preferably 1 to 15 wt.-% and preferably 2 to 10 wt.-%, each relative to the whole agent.

Sugar surfactants are known surface-active compounds, which include for example the sugar surfactant classes of alkylglucose esters, aldobionic acid amides, gluconamides (saccharic acid amides), glycerinamides, glyceringlycolipids, polyhydroxy fatty acid amide sugar surfactants and alkylpolyglycosides. Sugar surfactants preferred within the scope of the teaching according to the invention are alkylpolyglycosides and sugar amides as well as the derivatives thereof, in particular the ethers and esters thereof. The ethers are reaction products of one or more, preferably one, sugar hydroxy group with a compound containing one or more hydroxy groups, for example C₁₋₂₂ alcohols or glycols such as ethylene and/or propylene glycol, wherein the sugar hydroxy group can also have polyethylene glycol and/or polypropylene glycol residues. The esters are the reaction products of one or more, preferably one, sugar hydroxy group with a carboxylic acid, in particular a C₆₋₂₂ fatty acid.

Particularly preferred sugar amides satisfy the formula R′C(O)N(R″)[Z], in which R′ stands for a linear or branched, saturated or unsaturated acyl residue, preferably a linear unsaturated acyl residue, with 5 to 21, preferably 5 to 17, in particular 7 to 15, particularly preferably 7 to 13 carbon atoms, R″ stands for a linear or branched, saturated or unsaturated alkyl residue, preferably a linear unsaturated alkyl residue, with 6 to 22, preferably 6 to 18, in particular 8 to 16, particularly preferably 8 to 14 carbon atoms, a C₁₋₅ alkyl residue, in particular a methyl, ethyl, propyl, isopropyl, n-butyl, isobutyl, tert-butyl or n-pentyl residue, or hydrogen and Z stand for a sugar residue, i.e. a monosaccharide residue. Particularly preferred sugar amides are the amides of glucose, glucamides, for example lauroyl-methyl-glucamide.

Within the scope of the teaching according to the invention, the alkylpolyglycosides (APG) are particularly preferred sugar surfactants and satisfy preferably the general formula R¹O(AO)ₐ[G]ₓ, in which R′ stands for a linear or branched, saturated or unsaturated alkyl residue with 6 to 22, preferably 6 to 18, in particular 8 to 16, particularly preferably 8 to 14 carbon atoms, [G] stands for a glycosidically bonded sugar residue and x stands for a number from 1 to 10 and AO stands for an alkyleneoxy group, e.g. an ethyleneoxy or propyleneoxy group, and a stands for the average degree of alkoxylation from 0 to 20. Here, the group (AO)ₐ can also contain different alkyleneoxy units, e.g. ethyleneoxy or propyleneoxy units, wherein a is then the average overall degree of alkoxylation, i.e. the sum of the degree of ethoxylation and propoxylation. Where not stated differently or in more detail hereinafter, the alkyl residues Rⁱ of the APGs are linear unsaturated residues with the indicated number of carbon atoms.

APGs are non-ionic surfactants and represent known materials which can be obtained according to the relevant methods of preparative organic chemistry. The index value x indicates the degree of oligomerization (DP), i.e. the distribution of mono- and oligoglycosides, and stands for a number between 1 and 10. Whereas, in a given compound, x must always be an integer, and here above all can assume the values x=1 to 6, for a specific alkyl glycoside, the value x is an analytically determined calculated value which mostly represents a fractional number. Preferably, alkylglycosides with an average degree of oligomerization x of 1.1 to 3.0 are used. From a process engineering point of view, those alkylglyco sides where the degree of oligomerization is less than 1.7 and in particular lies between 1.2 and 1.6 are preferred. As glycosidic sugars there are used preferably xyloses, in particular, however, glucoses.

The alkyl or alkylene residue Rⁱ can be derived from primary alcohols with 8 to 18, preferably 8 to 14 carbon atoms. Typical examples are capronalcohol, caprylalcohol, caprinalcohol and undecylalcohol as well as the technical mixtures thereof, as accrue for example in the process of the hydrogenation of technical fatty acid methyl esters or in the process of the hydrogenation of aldehydes of Roelen's oxosynthesis.

Preferably, however, the alkyl or alkyl residue Rⁱ is derived from lauryl alcohol, myristyl alcohol, cetyl alcohol, palmoleyl alcohol, stearyl alcohol, isostearyl alcohol or oleyl alcohol. Furthermore, elaidyl alcohol, petroselinyl alcohol, arachidyl alcohol, gadoleyl alcohol, behenyl alcohol, erucyl alcohol as well as the technical mixtures thereof can be named.

Particularly preferred APGs are non-alkoxylated (a=0) and satisfy formula RO[G]ₓ, in which, as before, R stands for a linear or branched, saturated or unsaturated alkyl residue with 4 to 22 carbon atoms, [G] stands for a glycosidically bonded sugar residue, preferably glucose residue, and x stands for a number of from 1 to 10, preferably 1.1 to 3, in particular 1.2 to 1.6. Correspondingly preferred alkyl polyglycosides are for example C₈₋₁₀ and a C₁₂₋₁₄ alkyl polyglucoside with a DP of 1.4 or 1.5, in particular C₈₋₁₀ alkyl-1,5-glucoside and C₁₂₋₁₄ alkyl-1,4-glucoside.

The agent according to the invention can additionally contain one or more cationic surfactants (INCI quaternary ammonium compounds), conventionally in a quantity of from 0.001 to 5 wt.-%, preferably 0.01 to 4 wt.-%, in particular 0.1 to 3 wt.-%, particularly preferably 0.2 to 2 wt.-%, extremely preferably 0.5 to 1.5 wt.-%, for example 1 wt. %.

Preferred cationic surfactants are the quaternary surface-active compounds, in particular with an ammonium, sulfonium, phosphonium, iodonium or arsonium group, which are also known as antimicrobial active ingredients. By using quaternary surface-active compounds with an antimicrobial effect, the agent can be equipped with an antimicrobial effect or the antimicrobial effect already present possibly due to other ingredients, can be improved.

Particularly preferred cationic surfactants are the quaternary ammonium compounds (QACs) according to general formula (R^{I})(R^{II})(R^{III})(R^{IV})N^{—}X⁻, in which R^{I} to R^{IV} form identical or different C₁₋₂₂ alkyl residues, C₇₋₂₈ aralkyl residues or heterocyclic residues, wherein two, or in the case of an aromatic bonding, as in pyridine, even three residues, together with the nitrogen atom, represent the heterocycle, e.g. a pyridinium or imidazolinium compound, and X⁻ are halogenide ions, sulfate ions, hydroxide ions or similar anions. For an optimum antimicrobial effect, preferably at least one of the residues has a chain length of 8 to 18, in particular 12 to 16, C atoms.

QACs can be produced by reacting tertiary amines with alkylating agents, such as e.g. methyl chloride, benzene chloride, dimethyl sulfate, dodecyl bromide, but also ethylene oxide. The alkylation of tertiary amines with a long alkyl residue and two methyl groups is particularly easy, while the quaternization of tertiary amines with long residues and a methyl group can also be carried out with the help of methyl chloride under mild conditions. Amines which have three long alkyl residues or hydroxy-substituted alkyl residues are less reactive and are quaternized preferably with dimethyl sulfate.

Suitable QAVs are for example benzalkonium chloride (N-alkyl-N,N-dimethyl-benzylammonium chloride, CAS No. 8001-54-5), Benzalkon B (m,p-dichlorobenzyl-dimethyl-C₁₂-alkylammonium chloride, CAS No. 58390-78-6), benzoxonium chloride (benzyl-dodecyl-bis-(2-hydroxyethyl)-ammonium chloride), cetrimonium bromide (N-hexadecyl-N,N-trimethyl-ammonium bromide, CAS No. 57-09-0), benzetonium chloride, (N,N-dimethyl-N-[2-[2- [p-(1,1,3,3 -tetramethylbutyl)phenoxyethoxy] ethyl]-benzylammonium chloride, CAS No. 121-54-0), dialkyldimethyl ammonium chloride such as di-n-decyl-dimethyl ammonium chloride (CAS No. 7173-51-5-5), didecyldimethyl ammonium bromide (CAS No. 2390-68-3), dioctyldimethyl ammonium chloride, 1-cetylpyridinium chloride (CAS No. 123-03-5) and thiazoline iodide (CAS No. 15764-48-1) as well as mixtures thereof. Preferred QAVs are benzalkonium chlorides with C₈—C₁₈ alkyl residues, in particular C₁₂—C₁₄ alkyl-benzyl-dimethylammonium chloride. A particularly preferred QAV is cocopentaethoxy-methylammonium methosulfate (INCI PEG-5 cocomonium methosulfate; Rewoquat® CPEM).

To avoid possible incompatibilities of the cationic surfactants with the anionic surfactants, an anion-surfactant compatible cationic surfactant, and/or as little cationic surfactant as possible, is used, or in a specific embodiment of the invention, cationic surfactants are dispensed with entirely. In a preferred embodiment, the agent according to the invention is free from cationic and non-ionic surfactants.

To stabilize the amylases and possible further contained enzymes used, it is advantageous if an enzyme stabilizer is added to the cleaning agent according to the invention. Different suitable stabilizers are known from the prior art, such as for example saccharides, polyols, in particular propylene glycol or glycerol, furthermore borax, boric acids, boronic acids or the salts or esters thereof, in particular derivatives with aromatic groups, for example ortho, meta or para-substituted phenylboronic acids or even calcium salts such as calcium formiate, calcium acetate or calcium propionate.

However, the use of potassium salts has proven particularly advantageous. Here, both inorganic and organic potassium salts are used as the source of potassium ions. Preferred potassium salts are colorless. Among the inorganic potassium salts, those from the group of water-soluble halogenides, sulfates, sulfites, carbonates, hydrogencarbonates, nitrates, nitrites, phosphates and/or oxides are preferred, in particular halogenides and sulfates. The organic potassium salts which according to the invention can be used as a source for the potassium ions are in particular water-soluble potassium salts of carboxylic acids. The salts are preferably selected from the group consisting of formiate, acetate, propionate, citrate, malate, tartrate, succinate, malonate, oxalate, lactate, as well as mixtures thereof

Particularly advantageously, potassium chloride, potassium sulfate, potassium formiate, potassium acetate, potassium propionate and potassium lactate as well as mixtures thereof are provided. Potassium acetate and potassium chloride are quite particularly preferred.

In a preferred embodiment, the cleaning agent according to the invention therefore contains potassium ions. Preferably, the proportion of potassium ions in the overall composition is from 0.025 to 0.25 wt.-%, preferably 0.05 to 0.125 wt.-%.

Advantageously, in one embodiment of the invention, there are no builders which have calcium-precipitating properties present in the agent according to the invention. Correspondingly, an agent is preferred which in particular does not contain any carbonate-containing salts.

On the other hand, the use of other builders, preferably water-soluble builders, can be advantageous.

Organic builders which may be present in hand dishwashing detergents are for example the polycarboxylic acids used in the form of their sodium salts, wherein by polycarboxylic acids, those carboxylic acids are meant which have more than one acid function. For example, these are citric acid, adipic acid, succinic acid, glutaric acid, malic acid, maleic acid, fumaric acid, saccharic acids, amino carboxylic acids, nitrilotriacetic acid (NTA), methylglycinediacetic acid (MGDA) and derivatives as well as mixtures thereof. Preferred salts are those of polycarboxylic acids such as citric acid, adipic acid, succinic acid, glutaric acid, malic acid, saccharic acids and mixtures thereof

Further polymeric polycarboxylates are suitable as builders. These are for example the alkali metal salts of polyacrylic acid or of polymethacrylic acid, for example those with a relative molecular mass of 600 to 750,000 g/mol.

Suitable polymers are in particular polyacrylates which preferably have a molecular mass of 1,000 to 15,000 g/mol. In turn, from this group, the short-chained polyacrylates which have molar masses of 1,000 to 10,000 g/mol, and particularly preferably of from 1,000 to 5,000 g/mol are preferred on the basis of their superior solubility.

Furthermore, copolymeric polycarboxylates, in particular those of acrylic acid with methacrylic acid, and acrylic acid or methacrylic acid with maleic acid, are suitable. The polymers can also contain allyl sulfonic acids, such as allyloxy benzene sulfonic acid and methallyl sulfonic acid, to improve water solubility.

Preferably, however, soluble builders, such as for example citric acid, or acryl polymers with a molar mass of 1,000 to 5,000 g/mol, are used in the liquid hand dishwashing detergents.

The water content of the preferred liquid and aqueous hand dishwashing detergents is conventionally 15 to 90 wt.-%, preferably 20 to 85 wt.-%, in particular 30 to 80 wt.-%. The agent according to the invention can additionally contain one or more water-soluble organic solvents, conventionally in a quantity of from 0.1 to 30 wt.-%, preferably 1 to 20 wt.-%, in particular 2 to 15 wt.-%, particularly preferably 3 to 12 wt.-%, and extremely preferably 4 to 8 wt.-%.

Within the scope of the teaching according to the invention the solvent is used as required, in particular as a hydrotrope, viscosity regulator and/or additional cold stabilizer. It has a solubilizing power in particular for surfactants and an electrolyte as well as perfume and dye and thus contributes to the familiarization thereof, prevents the development of liquid-crystal phases and has a part in the formation of clear products. The viscosity of the agent according to the invention reduces as the quantity of solvent increases. However, too much solvent can have too strong an effect in reducing viscosity.

Particularly preferred, and particularly effective organic solvents, in respect of the stabilization of the enzymatic hand dishwashing detergents, are glycerol and 1,2-propylene glycol.

With regard to the manual application of the agents according to the invention, in a preferred embodiment, however, the use of organic solvents is dispensed with.

In addition to the above described solvents, for example also alkanol amines and alkyl benzene sulfonates with 1 to 3 carbon atoms are used in the alkyl residue as solubilizer, in particular for perfume and dyes.

In addition to the previously named components, the agents according to the invention can contain further ingredients. These include for example salts, additives for improving the draining and drying characteristics, for adjusting viscosity, for stabilizing auxiliaries and additives conventionally in hand dishwashing detergents, for example, UV stabilizers, perfume, pearlescing agents (INCI opacifying agents; for example glycol distearate, e.g. Cutina® AGS from Cognis, or mixtures containing same, e.g. Euperlane® from Cognis), dyes, corrosion inhibitors, preservatives (e.g. 2-bromo-2-nitropropane-1,3-diol (CAS 52-51-7), technically also called Bronopol, which for example is commercially available as Myacide® BT or as Boots Bronopol BT from Boots), organic salts, disinfectants, enzymes, pH adjusters as well as skin-sensation-improving or -soothing additives (e.g. dermatologically effective substances such as vitamin A, vitamin B2, vitamin B12, vitamin C, vitamin E, D-panthenol, sericerin, collagen partial hydrolyzate, different plant protein partial hydrolyzates, protein hydrolyzate fatty acid condensates, liposomes, cholesterol, plant and animal oils such as e.g. lecithin, soya oil, etc., plant extracts such as e.g. aloe vera, azulene, hamamelis extracts, algen extracts, etc., allantoin, A.H.A. complexes) which can be contained in quantities conventionally of not more than 5 wt. -%.

Cleaning agents according to the invention can contain salts, wherein in particular water-soluble salts are preferred. These include above all inorganic salts which are selected preferably from the group comprising colorless water-soluble halogenides, sulfates, sulfites, carbonates, hydrogencarbonates, nitrates, nitrites, phosphates and/or oxides of alkali metals, of alkaline-earth metals, of aluminum and/or of transition metals as well as mixtures thereof, particularly preferably from the group of halogenides and sulfates of alkali metals, as well as mixtures thereof, in particular from the group of sodium chloride, potassium chloride, sodium sulfate, potassium sulfate as well as mixtures thereof. Sodium chloride is particularly preferred.

Likewise, water-soluble organic solvents, for example formiate, acetate, propionate, citrate, malate, tartrate, succinate, malonate, oxalate, lactate as well as mixtures thereof, can be used according to the invention. Among the counterions, alkali, alkaline-earth or transition metal ions, aluminum ions and mixtures thereof are in turn preferred.

Furthermore, ammonium salts can also be used alone or together with other salts.

Further enzymes can be used in addition to the amylases used according to the invention. These include, for example, further amylases and in particular also proteases, but also further detergent or active cleaning enzymes. Suitable in particular are those from the class of hydrolases such as (poly)esterases, lipases, glycosyl hydrolases, hemicellulases, including in particular mannanases, xanthanlyases, pectinlyases (=pectinases), pectinesterases, pectatlyases, xyloglucanases (=xylanases), pullulanases and β-glucanases, cutinases, β-glucanases, oxidases, peroxidases, mannanases, perhydrolases, oxireductases and/or laccases. Reference is made to the relevant prior art in respect of washing or cleaning agents for further possible enzymes and enzyme preparations.

To further improve the draining and/or drying characteristics, the agent according to the invention can contain one or more additives from the group of surfactants, polymers and builders, conventionally in a quantity of from 0.001 to 5 wt.-%, preferably 0.01 to 4 wt.-%, in particular 0.1 to 3 wt.-%, particularly preferably 0.2 to 2 wt.-%, extremely preferably 0.5 to 1.5 wt.-%, for example 1 wt.-%, wherein as described above, calcium-precipitating builders are dispensed with as far as possible.

Polymers suitable as additives are in particular maleic acid acrylic acid copolymer Na salt (Sokalan® CP 5), modified polyacrylic acid Na salt (Sokalan® CP 10), modified polycarboxylate Na salt (Sokalan® HP 25), polyalkylene oxide, modified heptamethyl trisiloxane (Silwet® L-77), polyalkylene oxide, modified heptamethyl trisiloxane (Silwet® L-7608) as well as polyether siloxanes (copolymers of polymethyl siloxanes with ethylene oxide/propylene oxide segments (polyether blocks)), preferably water-soluble linear polyether siloxanes with terminal polyether blocks such as Tegopren® 5840, Tegopren® 5843, Tegopren® 5847, Tegopren® 5851, Tegopren® 5863 or Tegopren® 5878.

Builders suitable as additives are in particular polyaspartic acid Na salt, ethylene diamine triacetate cocoalkyl acetamide (Rewopol® CHT 12), methylglycinediacetic acid tri-Na salt (Triton® ES 9964) and acetophosphonic acid (Turpinal® SL). In the case of Monawet® MO-84 R2W, Tegopren® 5843 and Tegopren® 5863, mixtures with surfactants or polymeric additives show synergisms. The use of Tegopren types 5843 and 5863 is, however, less preferable in application on hard surfaces made of glass, in particular glassware, as these silicon surfactants can attach to glass. In a particular embodiment of the invention, the named additive is dispensed with.

Measured with a Brookfield LV DV II viscosimeter and spindle 25, at 20° C. and a shear rate of 30 min⁻¹, the viscosity preferred for the liquid agent according to the invention is in the range of from 10 to 10,000 mPa·s, preferably 600 to 6,000 mPa·s, in particular 1,000 to 5,000 mPa·s. The viscosity of the agent according to the invention can be increased by a thickener and/or reduced by a solvent.

Polymeric thickeners within the meaning of the present invention are polycarboxylates acting with a thickening effect as polyelectrolytes, preferably homo- and copolymerizates of acrylic acid, in particular acrylic acid copolymers such as acrylic acid methacrylic acid copolymers, and the polysaccharides, in particular heteropolysaccharides, as well as other conventional thickening polymers.

Suitable polysaccharides or heteropolysaccharides are polysaccharide gums, for example gum arabic, agar, alginate, carrageenan and the salts thereof, guar, guarana, tragacanth, gellan, ramsan, dextran or xanthan and the derivatives thereof, e.g. propoxylated guar, as well as mixtures thereof. Other polysaccharide thickeners, such as starches or cellulose derivatives, for example starches of the most different origin and starch derivatives, e.g. hydroxyethyl starch, starch phosphate esters or starch acetates or carboxymethylcelluloses or the sodium salt, methyl-, ethyl, hydroxyethyl, hydroxypropyl, hydroxypropyl-methyl or hydroxyethyl-methyl-cellulose or cellulose acetate thereof, can be used alternatively, preferably but also in addition to a polysaccharide gum.

A preferred polymeric thickener is the microbial anionic heteropolysaccharide xanthan gum, which is produced under aerobic conditions from Xanthomonas campestris and a few other species, with a molecular weight of from 2-15×10⁶ and can be obtained for example from Kelco under the commercial name Keltrol , e.g. as a cream-colored powder Keltrol® T (transparent) or as a white granular material Keltrol® RD (readily dispersible).

Acrylic acid polymers suitable as polymeric thickeners are for example high-molecular homopolymers of acrylic acid (INCI carbomer) which can be cross-linked with a polyalkenyl polyether, in particular an allylether of saccharose, pentaerythritol or propylene, which are also called carboxyvinyl polymers. Such polyacrylic acids can be obtained i.a. from BFGoodrich under the commercial name Carbopol®, e.g. Carbopol® 940 (molecular weight of approx. 4,000,000), Carbopol® 941 (molecular weight of approx. 1,250,000) or Carbopol® 934 (molecular weight of approx. 3,00,.000).

Particularly suitable polymeric thickeners are, however, the following acrylic acid copolymers: (i) copolymers of two or more monomers from the group of acrylic acid, methacrylic acid and their simple esters (INCI acrylates copolymer), formed preferably with C₁₋₄ alkanols, including, for example, the copolymers of methacrylic acid, butylacrylate and methylmethacrylate (CAS 25035-69-2) or of butylacrylate and methylmethacrylate (CAS 25852-37-3) and which for example can be obtained from Rohm & Haas under the commercial name Aculyn® and Acusol®, e.g. The anionic non-associative polymers Aculyn® 33 (cross-linked), Acusol® 810 and Acusol® 830 (CAS 25852-37-3); (ii) cross-linked high-molecular acrylic acid copolymers, including, for example, the copolymers of C₁₀₋₃₀ alkylacrylates cross-linked with an allyl ether of saccharose or of pentaerythritol, with one or more monomers from the group of acrylic acid, methacrylic acid and their simple esters (INCI acrylates/C10-30 alkyl acrylate crosspolymers) formed preferably with C₁₋₄ alkanols, and which for example are available from BFGoodrich under the commercial name Carbopol®, e.g. the hydrophobized Carbopol® ETD2623 and Carbopol® 1382 (INCI acrylates/C10-30 alkyl acrylate crosspolymers) as well as Carbopol® AQUA 30 (formerly Carbopol® EX 473).

The content of polymeric thickener is conventionally not more than 8 wt.-%, preferably between 0.1 and 7 wt.-%, particularly preferably between 0.5 and 6 wt.-%. In other embodiments, the proportion can also be between 1 and 5 wt.-%, preferably between 1.5 and 4 wt.-%, in particular preferably between 2 and 2.5 wt.-%.

In a special embodiment of the invention, the agent is, however, free from polymeric thickeners.

For stabilizing the agent according to the invention, in particular with a high surfactant content, one or more dicarboxylic acids and/or the salts thereof can be added, in particular a composition of Na salts of adipic acid, succinic acid and glutaric acid, as can be obtained e.g. under the commercial name Sokalan® DSC. Advantageously, these are used in quantities of from 0.1 to 8 wt.-%, preferably 0.5 to 7 wt.-%, in particular 1.3 to 6 wt.-% and particularly preferably 2 to 4 wt.-%.

A change in dicarboxylic acid (salt) content can—in particular in quantities above 2 wt.-%—contribute to a clear solution of the ingredients. Likewise, within specific boundaries, it is possible to influence the viscosity of the mixture due to this agent. Furthermore, this component influences the solubility of the mixture. This component is particularly preferably used in high surfactant contents, in particular in surfactant contents above 30 wt.-%.

However, their use can be dispensed with, with the result that the agent according to the invention is preferably free from dicarboxylic acids (salts).

Agents according to the invention can also contain elementary silver and/or a silver compound as antibacterial component.

Lactic acid also has the advantage that, like benzoic acid or also salicylic acid as a pH regulator and/or buffer substance, it can also support or reinforce the antibacterial effect of silver and/or of the silver compound.

The pH of the liquid agent according to the invention can, however, also be adjusted by means of conventional pH regulators, for example acids such as mineral acids or citric acid and/or alkalis such as sodium or potassium hydroxide, wherein—in particular with the desired skin and hand compatibility—a range of from 6.0 to 9.0, preferably 7.0 to 8.0 is preferred (measured undiluted and at 20° C.). For adjusting and/or stabilizing the pH, the agent according to the invention can additionally contain one or more buffer substances (INCI buffering agents), conventionally in a quantity of from 0.001 to 5 wt.-%, preferably 0.005 to 3 wt.-%, in particular 0.01 to 2 wt.-%, particularly preferably 0.05 to 1 wt.-%, extremely preferably 0.1 to 0.5 wt.- %, for example 0.2 wt.-%. Buffer substances which are complexers, or even chelating agents, are preferred. Particularly preferred buffer substances are citric acid or citrates, in particular sodium and potassium citrates, for example trisodium citrate-2·H₂O and tripotassium citrate·H₂O.

A liquid agent can furthermore contain hydrotropes. In this case, these are solubility enhancers. Suitable hydrotropes are for example urea, butyl glycol, or aliphatic short-chained anionic or amphoteric solubilizers.

In one embodiment, the liquid cleaning agent according to the invention is applied for cleaning hard surfaces, thus in particular for cleaning dirty crockery, in the form of a foam, either directly to the surface to be cleaned or on a sponge, a cloth, a brush or another, possibly wetted, cleaning auxiliary. A manually activated spray dispenser, in particular selected from the group comprising aerosol spray dispensers, even pressurized spray dispensers, pump spray dispensers and trigger spray dispensers, in particular pump spray dispensers, as available for example from the company Airspray, Taplast, Keltec or also the Daiwa Can Company, is especially suitable for producing the foam. In addition to trigger bottles, pump spray dispensers and trigger spray dispensers with a container made of polyethylene, polypropylene or polyethylene terephthalate are also suitable. Such trigger bottles are available for example from Afa-Polytec. The spray head is preferably equipped with a foam nozzle. In addition, the agent can also be filled using a suitable propellant (e.g. n-butane, a propane/butane mixture, carbon dioxide, nitrogen or a CO₂/N₂ mixture) in a corresponding aerosol spray dispenser. Such a spray dispenser is, however, less preferred.

Correspondingly, the agent according to the invention can be introduced in the form of a product from the agent according to the invention and a spray or foam bottle, in particular a pump foam dispenser.

For cleaning a hard surface by hand, the liquid cleaning agent according to the invention can be applied to the surface to be cleaned either directly, i.e. undiluted, for example by means of a sponge, and then removed with water.

Alternatively, the hand dishwashing detergent according to the invention can be diluted firstly with water at concentrations of 1:1 to 1:1000, and then the obtained cleaning solution brought into contact with the surface to be cleaned.

The present invention will be described in more detail below using examples. However, these examples are in no way limiting to the subject matter of the present invention and serve merely as illustration to a person skilled in the art. Embodiment examples

A formulation according to the invention E1 and a comparison formulation V1 were produced. E1 contained an amylase according to the invention (“Amylase 1”), whereas in V1, a conventional amylase (“Amylase 2,” Stainzyme 12 L) was used. The compositions are reproduced in the table below, wherein all quantities are in in wt.-% of the raw material used.

| | E1 | V1 |
|---|---|---|
| Fatty alcohol ether sulfate | 10 | 10 |
| Cocoamidopropyl betaine | 4 | 4 |
| Potassium acetate | 0.2 | 0.2 |
| Perfume | 0.2 | 0.2 |
| Preservative | 0.1 | 0.1 |
| Amylase 1 | 0.2 | — |
| Amylase 2 | — | 0.2 |
| Salts | 1 | 1 |
| Water | up to 100% | up to 100% |

Both formulations were checked with regard to their effect on dried starch at different temperatures:

Stainless steel plates (10×15 cm), on which in each case 1.2 g of a dried starch mixture (5 g of each of rice, maize, wheat, and potato starch swollen in 500 mL water) were soaked for 20 minutes in an aqueous solution of one of the two cleaning agent formulations (5 g/L), wherein in each case water of different temperatures (40° C./30° C./20° C.) was used. Then, the stainless steel plates were rinsed off under running water without wiping, and dried. Subsequently, starch removal was determined gravimetrically. The result is reproduced in the table below:

| | E1 | V1 |
|---|---|---|
| 40° C. | 97% | 93% |
| 30° C. | 66% | 39% |
| 20° C. | 65% | 8% |

Thus, compared with the prior art, the formulation according to the invention has clear performance advantages in particular at washing temperatures below 40° C.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A liquid cleaning agent for hard surfaces comprising an α-Amylase, wherein the α-Amylase is at least 89% identical to the sequence indicated in SEQ ID NO. 1 along the entire length thereof, and which has deletions in one or more of positions 180, 181, 182, 183 and 184 as numbered according to SEQ ID NO. 1.

2. The liquid cleaning agent according to claim 1, wherein the α-Amylase has deletions at at least two positions, selected from 180+181, 181+182, 182+183 and 183+184 as numbered according to SEQ ID NO. 1.

3. The liquid cleaning agent according to claim 1, wherein the α-Amylase has the deletions H183*+G184* as numbered according to SEQ ID NO. 1.

4. The liquid cleaning agent according to claim 1, wherein the α-Amylase furthermore has amino acid substitutions at one or more of positions 405, 421, 422 and 428 as numbered according to SEQ ID NO. 1.

5. The liquid cleaning agent according to claim 1, wherein the α-Amylase has the substitutions I405L; A421H, A422P and A428T as numbered according to SEQ ID NO. 1.

6. The liquid cleaning agent according to claim 1, wherein the agent has a content of α-Amylase of from 0.05 to 1.0 wt.-%.

7. The liquid cleaning agent according to claim 1, wherein the agent comprises one or more surfactants.

8. The liquid cleaning agent according to claim 7, wherein the agent comprises a surfactant combination of at least one anionic surfactant, and at least one amphoteric surfactant.

9. The liquid cleaning agent according to claim 7, wherein the agent comprises from 5 to 50 wt.-% surfactant.

10. The liquid cleaning agent according to claim 1, wherein the agent comprises potassium ions.

11. The liquid cleaning agent according to claim 10, wherein the proportion of potassium ions in the overall composition is 0.025 to 0.25 wt.-%.

12. The liquid cleaning agent according to claim 1, wherein the agent has a pH of from 6.0 to 9.0 (undiluted, 20° C.).

13. A method for cleaning hard surfaces comprising contacting the hard surface with a liquid cleaning agent according to claim 1.


SEQUENCE LISTING (1 sequence)

SEQ ID NO: 1 - protein, 485 aa, Artificial
  Hybridprotein
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGHGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGPQHDYIDHPDVIGWTREGDSSAAKSGLAALITDGPGGSKRMYAGLKNAGETWYDITGNRSDTVKIGSDGWGEFHVNDGSVSIYVQK